(19) United States
(12) Patent Application Publication
(10) Pub. No.: US 2013/0168463 A1
(43) Pub. Date: Jul. 4, 2013

(54) Fragrance slide sampler

(71) Applicants: Sven Dobler, Huntington, NY (US); Herve Ferrec, Epinay sur Seine (FR)

(72) Inventors: Sven Dobler, Huntington, NY (US); Herve Ferrec, Epinay sur Seine (FR)

(21) Appl. No.: 13/694,265

(22) Filed: Nov. 15, 2012

Related U.S. Application Data
(60) Provisional application No. 61/629,417, filed on Nov. 18, 2011.

(51) Int. Cl. A61L 9/12; B31D 1/00

(52) U.S. Cl. CPC A61L 9/12; B31D 1/00
USPC 239/34; 493/328

(57) ABSTRACT

A fragrance slide sampler incorporating a fragrance card being coated on one surface with a microencapsulated fragrance and one or more cover elements comprising a first side and a second side, one side being exposed to the user, and the second side being in contact with the microencapsulated fragrance card surface. When the fragrance card is moved, relative to the contiguous cover element, the fragrance card releases its aroma to frictional rubbing against the contiguous cover element, and releases its fragrance through the cover element for enhancing the aroma of the ambient area. Additional means for containing the fragrance sample are disclosed.

### CROSS REFERENCE TO RELATED APPLICATION

This non-provisional patent application claims priority to the provisional patent application having Ser. No. 61/629,417, filed on Nov. 18, 2011.

### FIELD OF THE INVENTION

This invention relates to fragrance samplers disturbed to consumers typically through insertion in magazines or used in direct mailings.

### BACKGROUND OF THE INVENTION

Traditionally fragrance samplers were dry pre-scented blotter cards that had to be individually overwrapped to contain the fragrance for use in direct mail or magazine advertising. Beginning in the late 1970's, the micro-encapsulated Scentstrip® style magazine and direct mail insert was introduced. The Scentstrip insert is described in U.S. Pat. No. 5,093,182 to Ross. This product was produced on wide web offset printing equipment and therefore offered significant cost efficiencies for mass marketing. A major drawback of this system is that the microencapsulated surface must be scratched or abraded in order to release the fragrant oils. This typically involves the consumer scraping their fingernail across the microencapsulated surface, which results in microencapsulation particles and fragrant oils deposited under the fingernails. Mailing of micro-encapsulated fragrances also results in fragrances inadvertently released during transit and permeating the magazine or direct mailer. Other devices have attempted to address these problems, for instance, U.S. Pat. No. 4,889,755 describes a device which uses a microencapsulated fragrance that is bound to both a sampling sheet and internally to the device, requiring the user to break the bond by removing the sampling sheet in order to sample the fragrance. U.S. Pat. No. 4,186,743 describes the use of microcapsules on a pressure sensitive adhesive between two surfaces on a sanitary napkin. When a cover layer is removed, capsules are broken and the fragrance is released. U.S. Pat. No. 4,487,801 describes the use of a non-pressure sensitive adhesive layer between two surfaces, the layer having fragrance containing microcapsules therein. Upon separation of the two surfaces, the adhesive and the microcapsules are ruptured, releasing the fragrance. U.S. Pat. No. 4,720,417 shows a similar article in which the two surfaces are coated paper surfaces.

The instant invention overcomes these shortcomings by providing a device that covers the micro-encapsulated fragrance until use, and allows the consumer to release the fragrance by simply by moving an element of the device.

The instant invention also offers advertisers space to present printed words or designs. A Fragrance Slide Sampler, in accordance with the present invention, delivers a fragrance sample to a consumer using an inexpensive device that will not release the fragrance until desired by the user. The device contains microencapsulated fragrance, but unlike other devices with microencapsulated fragrance it allows the user to release the fragrance without depositing microencapsulation debris or fragrance on the fingers. Its features also include simplicity in both use and manufacture.

### SUMMARY OF THE INVENTION

The concept of this invention is to provide a fragrance sampler that includes a fragrance card that incorporates one or more fragrance card elements, with at least one of their surfaces being coated with a microencapsulated fragrance. One or more cover elements comprise a first side and a second side, with a first side being visible to the user and exposed to the atmosphere, while the second side is in contact with the micro encapsulated surface. The cover element substantially conceals the fragrance card from the atmosphere and from view. There is a frame attached to the cover element, providing the space between the second side and the said frame, for containment of said fragrance card therein, whereby when said microencapsulated surface is in contact with the second side of the cover element, such that when a means provided for moving the fragrance card relative to one or more of the covered elements is applied, it thereby moves the fragrance card and thereby exposes the fragrance card to the atmosphere of the user, and the microencapsulated fragrance frictionally releases its fragrance oils to the atmosphere, to provide a pleasant ambient aroma.

It is, therefore, the principal object of this invention to provide a fragrance slide sampler, that includes basically a cover element, having first and second sides, one of the sides being coated with a microencapsulated surface, and a movable fragrance card, as coated, provided for sliding contiguously against the cover element, to release the fragrance aroma when the fragrance card rubs against the cover element to frictionally open the micro encapsulated oils, and to provide for their release to the ambient atmosphere.

These and other objects may become more apparent to those skilled in the art upon review of the summary of the invention as provided herein, and upon undertaken a study of the description of its preferred embodiments.

### BRIEF DESCRIPTION OF THE DRAWINGS

In referring to the drawings:

FIG. 1A discloses the fragrance card, covered with an microencapsulated fragrance;

FIG. 1B shows the cover element and its frame;

FIG. 1C shows the fragrance card located within the folded cover element;

FIG. 1D shows the fragrance card partially pulled out from the cover elements of the frame;

FIG. 1E is a sectional view taken along the line 1e-1e of FIG. 1c;

FIG. 1F is a sectional view taken along the line 1f-1f of FIG. 1d;

FIG. 2A shows the fragrance card for the preferred embodiment of this invention;

FIG. 2B shows the opened frame for holding the fragrance card of this invention;

FIG. 2C shows the assembled preferred embodiment for the fragrance slide sampler;

FIG. 2D shows the fragrance card being partially retracted from the frame for the preferred embodiment of the fragrance slide sampler;

FIG. 2E is a cross sectional view taken along the line 2e-2e of FIG. 2c;

FIG. 2F is a cross sectional view taken the line 2f-2f of FIG. 2d;

FIG. 3A shows the open frame of one embodiment of this invention;

FIG. 3B shows a frontal view of the assembled embodiment of FIG. 3;

FIG. 3C shows a back view of the assembled embodiment of FIG. 3;

FIG. 3D shows a prospective view of the embodiment of FIG. 3 with the fragrance card in the in retracted position;

FIG. 3E shows a prospective view of the embodiment of FIG. 3 with the fragrance card in the in extended position;

FIG. 3F is a cross sectional view taken along the line 3h-3h of FIG. 3D;

FIG. 3G is a cross sectional view taken along the line 3i-3i of FIG. 3E;

FIG. 4A shows a forward view of a fragrance card containing a fragrance pouch;

FIG. 4B shows a cross section taken along the line of 4g of FIG. 4A;

FIG. 4C shows the cross section of FIG. 4B while the fragrance pouch is opened by the user;

FIG. 5A shows a forward view of a fragrance card containing a fragrance treated material; and

FIG. 5B shows a cross section taken along the line of 5h of FIG. 5A.

### DESCRIPTION OF THE PREFERRED EMBODIMENT

Numerous embodiments, features, and advantages of the present invention will be readily apparent to those of ordinary skill in the art upon a reading of the following detailed description of the presently preferred, but nonetheless illustrative, embodiment of the present invention when taken in conjunction with the accompanying drawings. Before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, the phraseology and terminology employed herein are for the purpose of description and should not be regarded as limiting.

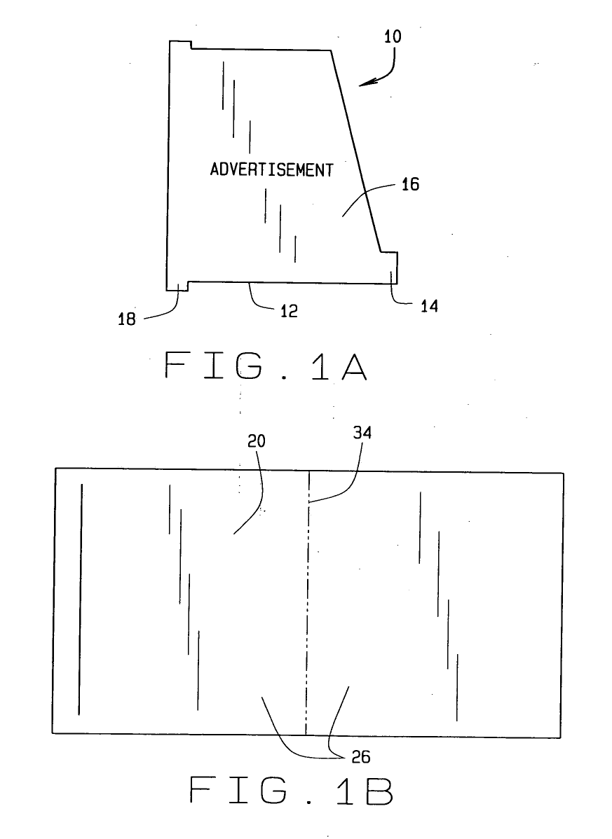

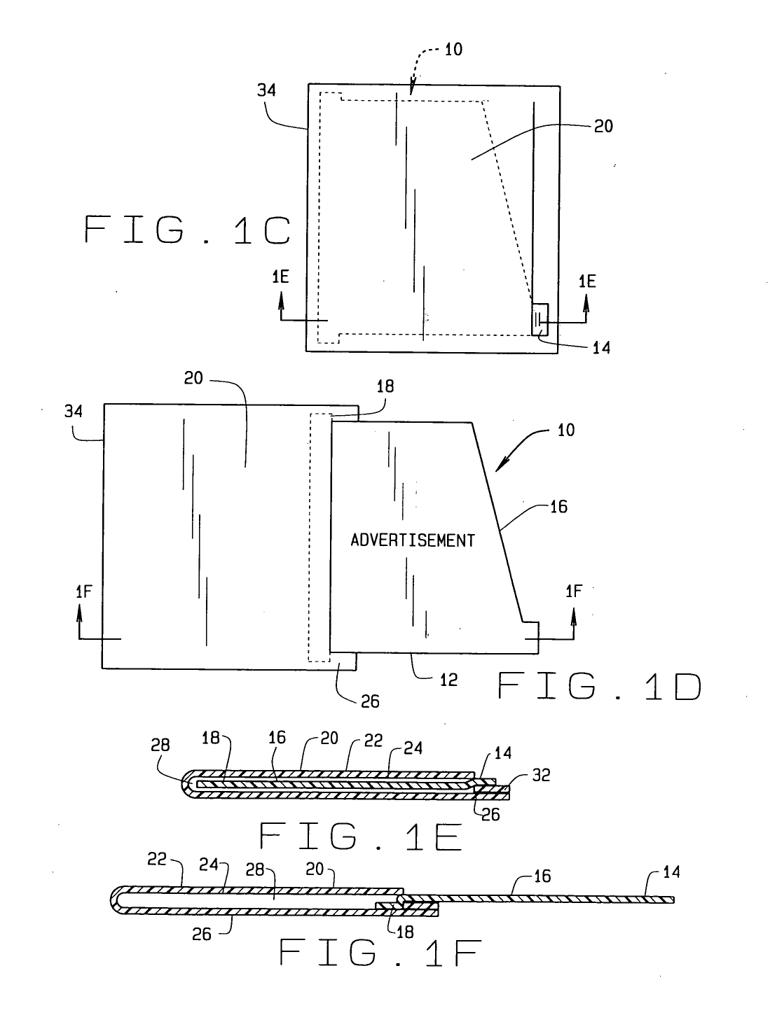

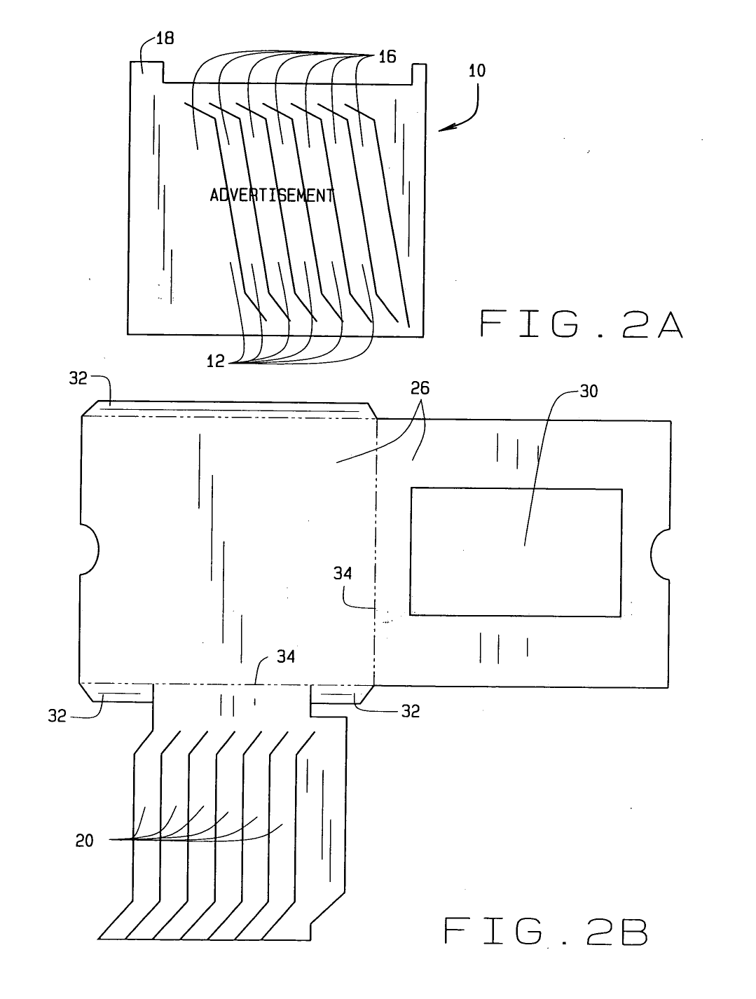

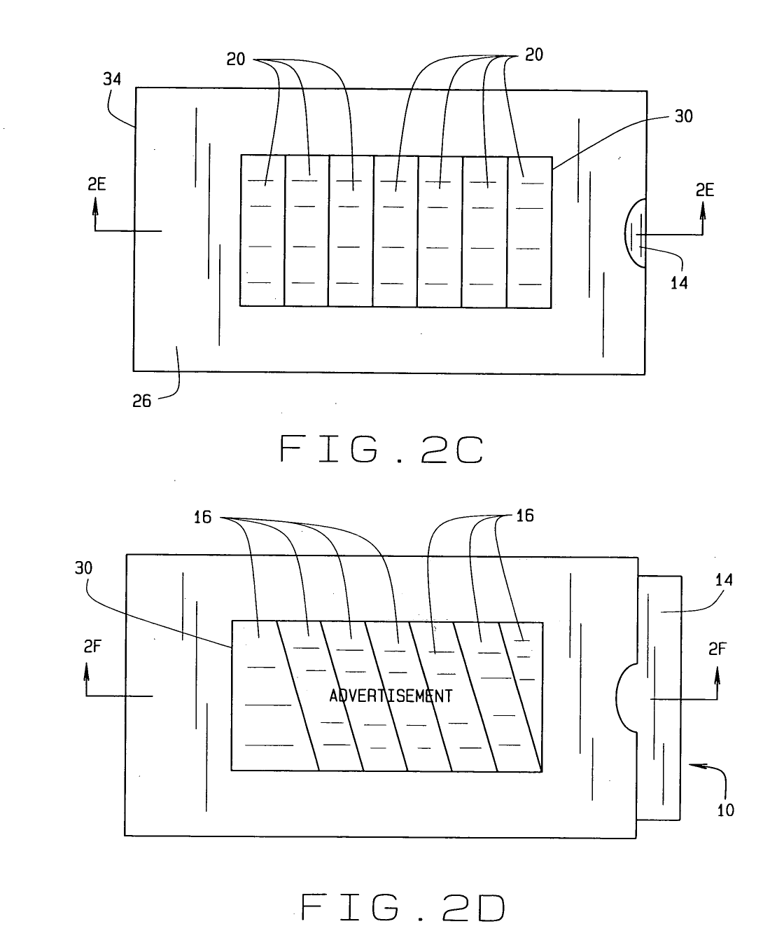

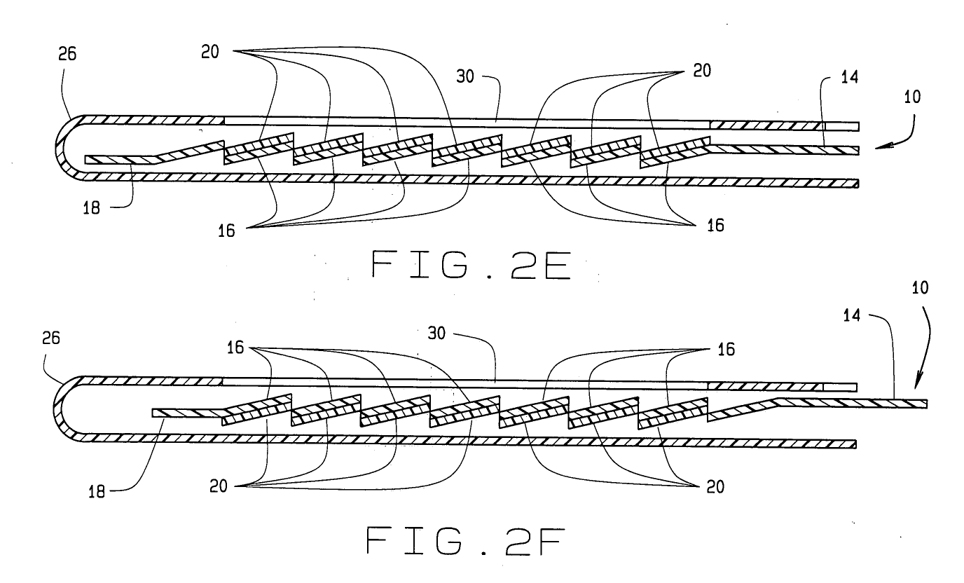

As illustrated in FIGS. 1A-1F and 2A-2F, the Fragrance Slide Sampler is a fragrance sampling device that is comprised of a fragrance card 10, a microencapsulated fragrance 16, a cover element 20, and a frame 26. The fragrance card 10 further comprises a portion of the fragrance card that may be extended from the device, hereafter referred to as the fragrance card element 12. The fragrance card element 12 contains a microencapsulated fragrance 16 on at least one surface, preferably the surface that will be exposed to the user. The microencapsulated fragrance 16 and fragrance card element 12 are in contact with, and concealed by, the cover element 20 in the retracted position. The fragrance card 10 further comprises a means for moving the fragrance card 14 and the fragrance card element 12 from the retracted to the extended positions or out from behind the cover element, thereby exposing the fragrance card element 12 and microencapsulated fragrance 16 to the atmosphere and to the user. The fragrance card 10 may comprise one or more fragrance card elements 12, each containing a microencapsulated fragrance 16 on at least one surface, and each concealed by a corresponding cover element 20. The one or more fragrance card elements 12 are positioned behind a corresponding cover element 20 while in the retracted position, thereby concealing and protecting the microencapsulated fragrance 16 from the atmosphere until use, and preventing the inadvertent release of the fragrance. As the microencapsulated fragrance 16 is in contact with the cover element 20 in the retracted position, movement of the fragrance card from the retracted position to the extended position creates friction between the microencapsulated fragrance 16 and the cover element 20 which assists in releasing the fragrant oils. It is not necessary that the entire fragrance card 10 or fragrance card element 12 be exposed. The fragrance card 10 may also contain a means for limiting movement 18 to prevent the fragrance card 10 from being completely separated from the frame 26 and cover element 20. The fragrance card 10 and/or fragrance card elements 12 may also contain printed words or deigns to deliver a message or advertisement which is revealed when the fragrance card is moved into the extended position. After use the fragrance card 10 may be returned to the retracted position and the process repeated as long as fragrant oils remain. The Fragrance Slide Sampler also comprises a frame 26 which together with the cover elements 20 provide a space 28 to contain the fragrance card. The frame 26 positions the fragrance card 10 and cover elements 20 such that the cover elements 20 conceal and are in contact with the fragrance card elements 12 and the microencapsulated fragrance 16 in the retracted position.

FIG. 1A-1F illustrates one embodiment of a Fragrance Slide Sampler that comprises a fragrance card 10 with a single fragrance card element 12, microencapsulated fragrance 16, a single cover element 20, and a frame 26. FIG. 1C illustrates the fragrance card element 12 in the retracted position, while FIG. 1D illustrates the fragrance card element 12 in the extended position. The Fragrance Slide Sampler of FIGS. 1A and 1B comprises a fragrance card 10 with a fragrance card element 12 with a microencapsulated fragrance 16 on one surface of the fragrance card element 12. The fragrance card element 12 refers to the portion of the fragrance card 10 which is exposed to the user when the fragrance card is moved from the retracted to the extended position. In FIG. 1A, the fragrance card element 12 comprises nearly the entire fragrance card 10. The fragrance card 10 may also comprise a means for moving the fragrance card 14, and a means for stopping 18 the fragrance card 10 from becoming separating from the frame 26 and cover element 20. See FIG. 1D. FIG. 1E which shows a single cover element 20 comprising a first side 22 and second side 24. The first side 22 of the cover element faces the user while the second side 24 is in contact with the microencapsulated fragrance 16. Also shown is a frame 26 which together with the cover element 20 forms a space 28 for the fragrance card 10. Show in FIG. 1C, is the fragrance card 10 in the retracted position, where the fragrance card positioned in the space 28 between the cover element 20 and the frame 26 such that the microencapsulated fragrance 16 is in contact with the second side 24 of the cover element 20. When a user wishes to sample the fragrance, the fragrance card 10 is pulled from the retracted position as shown in FIG. 1C, to the extended position, as shown in FIG. 1D, exposing the fragrance card element 12 to the atmosphere and to the user. Movement from the retracted position to the extended position creates friction between the second side 24 of the cover element and the microencapsulated fragrance 16 thereby releasing the fragrant oils. The fragrance card 10 is positioned in the retracted position while in transport to the consumer or user thereby concealing the microencapsulated fragrance from the atmosphere and from view. After receiving the Fragrance Slide Sampler the user of the device will pull the fragrance card 10 using a means of moving the fragrance card 14, for example a tab, into the extended position thereby exposing the fragrance card element 12 to the user and releasing the fragrant oils. It is not necessary that the entire fragrance card 10 or fragrance card element 12 be exposed. The fragrance card may also contain a means for limiting its movement 18 to prevent the fragrance card from being completely separated from the frame 26 and cover element 20. After use the fragrance card may be returned to the retracted position and the process repeated as long as fragrant oils remain.

FIGS. 2A through 2F illustrates a preferred embodiment of a Fragrance Slide Sampler whereby the Sampler includes a fragrance card with a plurality of fragrance card elements 12 and a corresponding number of cover elements 20. FIG. 2C illustrates the fragrance card in a retracted position while FIG. 2D illustrates the fragrance card in the extended position. The Fragrance Slide Sampler of FIG. 2 comprises a fragrance card 10 with a means for moving the fragrance card 14, from the retracted to the extended position to expose the fragrance card elements 12 and microencapsulated fragrance 16 to the user. FIG. 2 shows a plurality of cover elements 20 corresponding to the plurality of fragrance card elements 12. Each cover element comprises a first 22 and second side 24. The cover elements first side 22 faces the user while the cover elements second side 24 is in contact with the microencapsulated fragrance 16. Also shown is a frame 26 which together with the cover element 20 forms a space 28 for the fragrance card 10, and positions the fragrance card 10, fragrance card elements 12 and cover elements 20 relative to one another. In the retracted position, the fragrance card elements are positioned in the space 28 between the cover element 20 and the frame 26 such that the microencapsulated fragrance 16 is concealed by, and in contact with, the second side 22 of the corresponding cover element 20. Upon use, the fragrance card 10 and fragrance card elements 12 are moved from the retracted position to the extended position, exposing the fragrance card elements 12 to the atmosphere and to the user. Movement from the retracted position to the extended position creates friction between the second side 24 of the cover element and the microencapsulated fragrance 16 which aid in releasing the fragrant oils. The fragrance card is positioned in the retracted position while the device is in transport to the user thereby concealing the microencapsulated fragrance from view and from the atmosphere. After receiving the Fragrance Slide Sampler the consumer will use the device by pulling the fragrance card 10 from the retracted to the extended position exposing the fragrance card elements 12 and microencapsulated fragrance 16 to the consumer and releasing the fragrant oils. It is not necessary that the entire fragrance card or fragrance card element be exposed. The fragrance card may also contain a means for stopping its movement 18 to prevent the fragrance card 10 from becoming completely separated from the frame 26 and the cover elements 20. After use the fragrance card may be returned to the retracted position and the process repeated as long as fragrant oils remain.

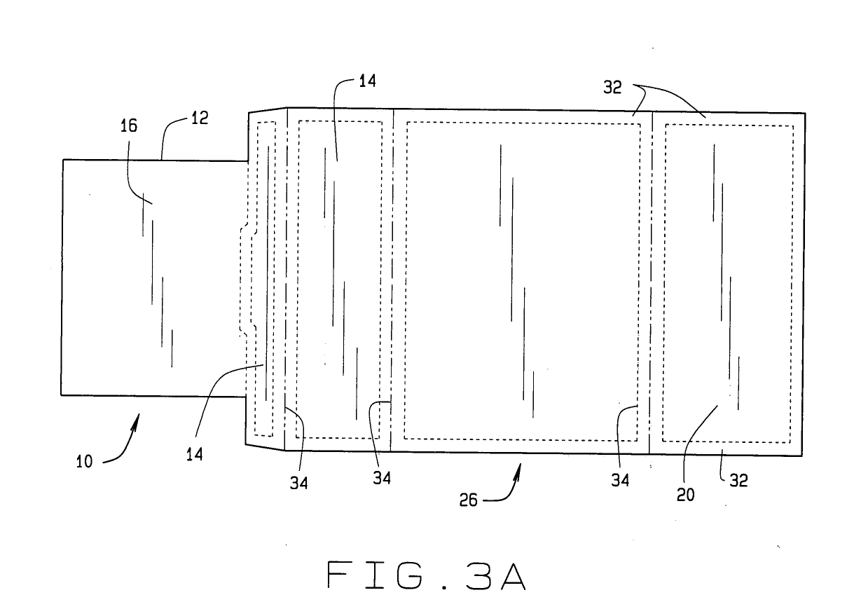

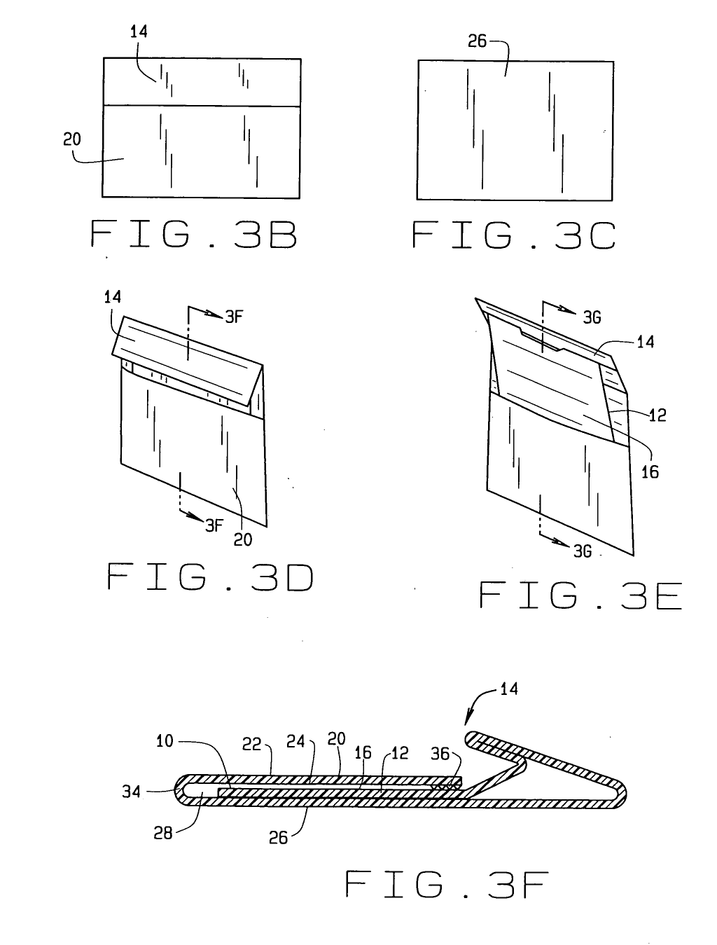

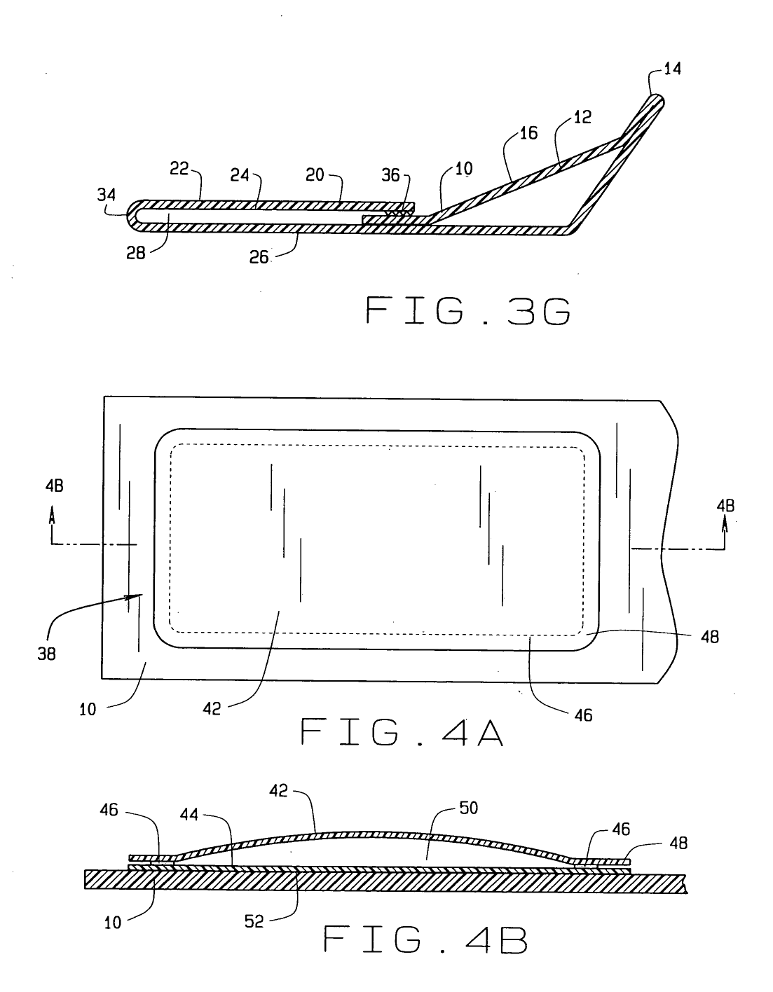

FIGS. 3A-3G illustrates yet another embodiment of a Fragrance Slide Sampler whereby the invention may be manufactured from a single piece of continuous stock material. The stock material is preferably a flexible material, by way of example, card stock, coated or uncoated paper, synthetic paper, laminated combinations of film and paper substrates, or plain plastic materials. The embodiment of FIG. 3 comprises a frame 26, a single fragrance card 10, and a single cover element 20 which conceals and protects the fragrance card 10 until use. The embodiment also comprises a means for moving the fragrance card 14 which is attached to both the frame 26 and the fragrance card 10 and folds towards the cover element 20 when the fragrance card 10 is in the retracted position. The fragrance card 10 and fragrance card element 12 comprise a microencapsulated fragrance 16 on at least one surface. As described in other embodiments the fragrance card element 12 refers to the portion of the fragrance card 10 which is exposed to the user when the fragrance card 12 is moved from the retracted to the extended position. The fragrance card element 12 of FIG. 3 comprises the entire or nearly the entire fragrance card 10.

A non-limiting example of the embodiment in its unassembled form is show in FIG. 3A. The embodiment is preferably cut from a larger piece of stock material and assembled by folding at predetermined positions 34. Adhesive 32 may be applied to any one or more surfaces to secure the frame 26 in the folded configuration, as illustrated by example in FIGS. 3B-3E. A microencapsulated fragrance 16 may be applied to one or more the surfaces of the fragrance card 10 at any stage in the manufacturing or assembly process, and by any effective means including, flexographic print, silk screen print, spray deposit, or extrusion, and allowed to dry in the ambient air or upon heating.

As shown in FIG. 3, the embodiment comprises a means for moving the fragrance card 14 that is flexibly attached to the fragrance card 10 and the frame 26, the flexible attachments arising from the embodiment being manufactured from a continuous piece of flexible stock material. The flexible material allows a hinge like movement between the means for moving the fragrance card 14 and both the frame 26, and the fragrance card 10, such that when the user opens or unfolds the means for moving the fragrance card 14, the hinge like movement extracts the fragrance card 10 from behind the cover element 20 as shown in FIG. 3E. This movement creates friction between the second side 24 of the cover element 20 and the microencapsulated fragrance 16 thereby releasing the fragrant oils.

FIGS. 3F-G illustrates the embodiment in cross-section and shows a frame 26, and a cover element 20, which together form a space 28 to contain the fragrance card 10. FIGS. 3F-G also illustrates a cover element 20 comprising a first side 22 and second side 24. The first side 22 of the cover element 20 faces the user while the second side 24 is in contact with the microencapsulated fragrance 16. FIG. 3E illustrates the fragrance card 10 in the retracted position and means for moving the fragrance card 14 in a folded or closed position, while FIG. 3G illustrates the fragrance card 10 in an extended position and the means for moving the fragrance card 14 in an unfolded or open position. When a user wishes to sample the fragrance, the means for moving the fragrance card 14 is opened or unfolded, and the fragrance card 10 is pulled from the retracted position, as shown in FIG. 3E, to the extended position, as shown in FIG. 3G, thereby exposing the fragrance card 10 and/or fragrance card element 12 to the atmosphere and to the user. Movement from the retracted position to the extended position creates friction between the second side 24 of the cover element 20 and the microencapsulated fragrance 16 thereby releasing the fragrant oils.

The embodiments of the Fragrance Slide Sampler disclosed herein may also contain a means for increasing friction between the second side 24 of the cover element 20 and the fragrance card 10 or microencapsulated fragrance 16. One example of a means for increasing friction is to minimize the space 28 between the frame 26 and the second side 24 of the cover element 20, thereby increasing tension on the fragrance card 10 in the retracted position. Another example of a means for increasing friction is by directing the movement of the fragrance card 10 against the second side 24 of the cover element 20 when moved from the retracted to the extended position. As illustrated in the FIGS. 1E-F, 2E-F, and 3F-G, the frame 26 together with one or more cover elements 20, may form a path that the fragrance card 10 must follow from the retracted position to the extended position which directs the fragrance card 10 against the second side 24 of the cover element 20. Directing movement of the fragrance card 10 against the second side 24 of the cover element 24 will increase friction between the microencapsulated fragrance 16 and the second side 24 of the cover element 20, thereby rupturing the microcapsules and releasing fragrance.

FIGS. 1E-F illustrates the fragrance card being forced into an angular path against the edge of the cover element 20 by the frame 26 when moved from the retracted position to the extended position. FIGS. 2E-F, illustrates the one or more fragrance card elements 12 moving along an angular path into the plane of the one or more cover elements 20, thereby increasing friction. FIGS. 3F-G, illustrate the fragrance card 10 being pulled by a means for moving the fragrance card 14 along an angular path against the cover element 20, thereby increasing friction against the microencapsulated fragrance 16. By angling the path of fragrance card 10 against the one or more cover elements 20, pressure against the microencapsulated fragrance 16 increases, as does rupture of the microcapsules and release of the fragrant oils.

Another means for increasing friction is by optionally incorporating an abrasive element 36 into the second side of the cover element as illustrated in FIG. 3F-G The second side 24 of the cover element 20 of the embodiment of FIG. 3, or any embodiment containing a microencapsulated fragrance 16, may also contain an abrasive element 36, to aid in creating friction and releasing the fragrant oils. This abrasive element 36 may be any abrasive material, by way of example sand paper that has been secured to the second side 24 of the cover element 20 or more preferable, an abrasive element 36 may be deposited during manufacture, through offset printing, flexographic printing, silk screen, embossing or texturing in other means. The embodiment of FIG. 3 may or may not comprise a means for limiting movement 18.

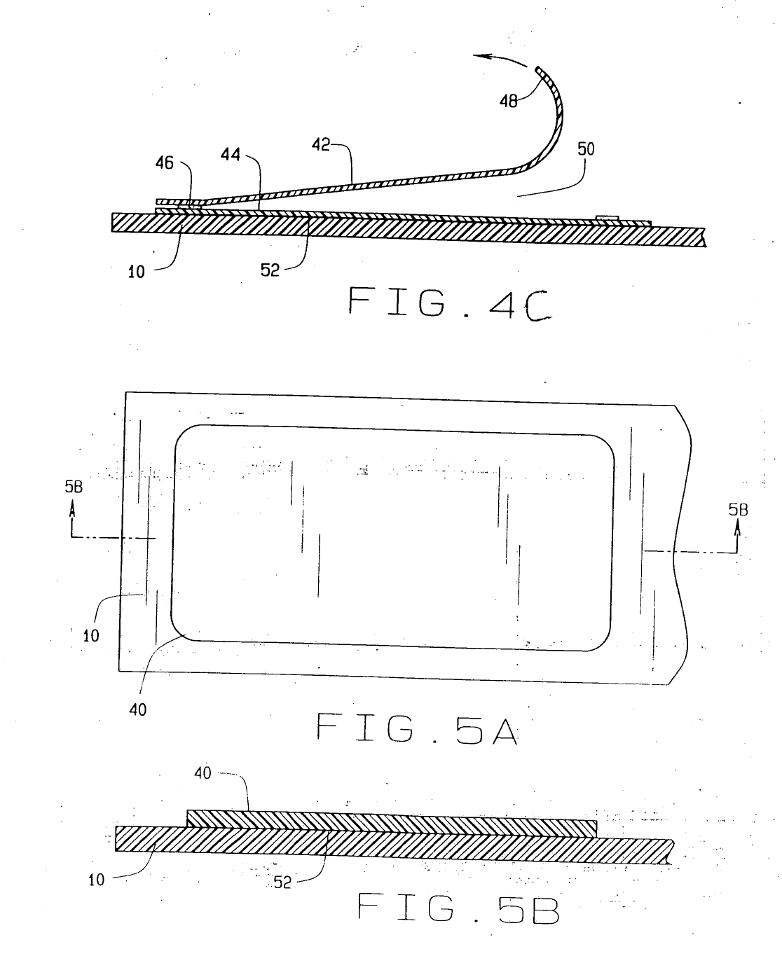

In yet another embodiment is a Fragrance Slide Sampler similar to that illustrated in FIGS. 3A-3G, with the exception that the microencapsulated fragrance 16 is replaced with a contained fragrance. Non-limiting examples of contained fragrances include a fragrance pouch 38, which may be an envelope-like device, comprised of thin plastic or foil walls, enclosing a liquid, or gel fragrance as illustrated in FIG. 4A-4C. Another example of a contained fragrance is a fragrance treated material 40, as illustrated in FIG. 5A-B. Examples of fragrance treated materials 40 include card, paper, gel, or absorbent material that may be coated or impregnated with a fragrance. The fragrance pouch 38 may optionally contain a means for the user to easily open the fragrance pouch. By way of example, FIGS. 4A and 4B illustrates a fragrance pouch 40 comprised of a first pouch wall 42 and a second pouch wall 44, attached to one another near their periphery, by way of a non-permanent adhesive 46 to form an impervious seal. The fragrance pouch 38 may optionally possess a means for opening 48. After extracting the fragrance card 10 as described for FIG. 3D-3G, the user may grasp the means for opening 48 and break the non-permanent adhesive 46, thereby separating the first pouch wall 42 from the second pouch wall 44 and releasing the fragrance 50, as illustrated in FIG. 4C. Alternatively, the contained fragrance may consist of fragrance treated material 40, as illustrated in FIG. 5A-5B. After extracting the fragrance card 10 as described in FIG. 3D-3G, the fragrance treated material 40 will freely release its fragrance upon exposure to the ambient air.

The embodiment of FIG. 3A-G and 4A-C or the embodiment of FIGS. 3A-G and 5A-B may be manufactured in a manner similar to that described for the embodiment of FIG-3A-3G, with the exception that the contained fragrance will be attached to the fragrance card 10 instead of applying the microencapsulated fragrance 16. The contained fragrance, whether in the form of a fragrance pouch 38 or a fragrance treated material 40, may be affixed to the fragrance card 10 using any acceptable means, including permanent or non-permanent adhesives 52. The use of a non-permanent adhesive is preferable in that it will also allow the user to easily remove the contained fragrance from the fragrance card 10 to enjoy at a later time if desired. In addition, the contained fragrance, whether a fragrance pouch 38 or a treated material 40 may be manufactured on or attached to materials containing a non-permanent adhesive or by way of example pressure sensitive adhesive technology and applied to the fragrance card at any time during manufacture or assembly.

The term non-permanent adhesive as used herein refers to any adhesive system or process that provides a secure impermeable attachment but allows the user to reverse the attachment with reasonable effort. The nature of the non-permanent adhesive including, its composition and manner of activation, are not critical as long as the following properties are met: the adhesive has an adhesive strength such that the tensile strength of the adhesive is not greater than the surround materials, that breaking the adhesive will not compromise the surrounding materials, and the adhesive forms an impervious seal prior to breaking such that the liquid or gel is contained. It is not critical for re-adhesion to occur after the seal has been broken. Examples of non-permanent adhesives are well known in the art and include those used in pressure sensitive adhesive technology widely used in labeling as well as those described in U.S. Pat. Nos. 4,181,635, 5,961,770 and 6,414,073, hereby incorporated by reference in their entirety.

Regardless of the particular embodiment, the fragrance card 10 is positioned in the retracted position while in transport to the consumer or user thereby concealing the microencapsulated fragrance or contained fragrance from the atmosphere and from view. After use the fragrance card may be returned to the retracted position and the process repeated as long as fragrant oils remain.

The Fragrance Slide Sampler may contain printed words or deigns, for example advertisements. The fragrance card 10 or fragrance card elements 12 may reveal printed words or deigns, and/or, conceal may reveal printed words or deigns, from the user when moved from the retracted to the extended position.

Microencapsulation is well known in the art. Non-limiting Examples may be found in U.S. Pat. No. 5,093,182, to Ross, hereby incorporated by reference in its entirety. Essentially, the microencapsulation fragrance of the present invention is comprised of a sustained release perfume-containing coating obtained from a non-aqueous solution or dispersion of a selected polymeric material such as ethyl cellulose in fragrant oil. A perfume-containing composition may be applied as a coating to the fragrance card. The microencapsulation fragrance may be applied over the same area or on top of a printed image. The coating may be prepared by dissolving a selected polymer, preferably ethyl cellulose in fragrance oil in the absence of an aqueous phase, with the amount and properties of the polymer being controlled to produce a required viscosity and tackiness to facilitate its application as a coating. The fragrance may comprise natural or synthetic essential oils which include a variety of fragrance components differing from one another in their chemical functionality, molecular weight, and volatility. Such oils are available in the form of specific blends that provide a desired aroma. Fixatives such as isopropyl myristate, DEP diethylphthalate), methylglucose ether, benzyl benzoate, or BHT (butylated hydroxytoluene) may also be included in such oils. Further examples of teachings of microencapsulated fragrance include U.S. Pat. No. 5,391,420 to Bootman, U.S. Pat. No. 5,161,688 to Muchin, and U.S. Pat. No. 5,622,263 to Greenland, which may be adapted to fragrance card of the present invention, and are hereby incorporated by reference in their entirety.

The Inventors also envision a manufacturing process whereby the microencapsulated fragrance 16 may be applied to the either or both the fragrance card 10 and the second side 24 of the cover element 20. The invention may then be assembled before the microencapsulated fragrance 16 becomes affixed to the surface. In this manner, the microencapsulated fragrance 16 will adhere to both fragrance card 10, or fragrance card element 12, and the second side 24 of the cover element 20. Upon extending the fragrance card 10, the microencapsulated fragrance 16 that is adhered to both opposing surfaces will rupture and release fragrant oils.

The fragrance card 10 may be may be made of paper, paperboard, polymers, or preferable cardstock, and may comprise one or more fragrance card elements 12. A fragrance card element 12 is a portion of the fragrance card that is exposed to the user when the fragrance card 10 is moved from the retracted to the extended position. The fragrance card may comprise a single fragrance card element 12 as in FIG. 1, which is extended from behind a cover element 20 by the user, or may comprise multiple fragrance card elements 12, as in FIG. 2, which are extended from behind multiple cover elements 20 by the user. The fragrance card elements 12 are treated, impregnated, or preferably coated with microencapsulated fragrance 16 on at least one surface, preferably the surface that is in contact with the cover element second side 24, when in the retracted position, and exposed to the user when in the extended position. The fragrance card 10 may also comprise or attach to a means for moving 14 the fragrance card, for example a tab, by which the user will grasp or pull the fragrance card from the retracted position to the extended position. The fragrance card 10 may also contain or attach to a means for stopping 18 the fragrance card 10 from becoming separated from the frame 26 and cover element 20. The fragrance card 10 or fragrance card elements 12 may also contain printed words or deigns, for example advertising, indicating the name of the product or any message or branding that the sponsor desires.

The cover element 20 may be may be made of paper, paperboard, polymers, or preferable cardstock, and functions to cover the fragrance card elements 12 while in the retracted position. The cover element elements 20 may be attached to, or integrated with the frame 26, such that the term cover element refers to the material that covers the fragrance card elements 12 in the retracted position. The cover element together with the frame 26 forms a space 28 which the fragrance card 10 occupies in the retracted position. The cover element 20 comprises a first side 22 and a second side 24. The first side 22 is visible to the user and the second side 24 is in contact with the microencapsulated fragrance 16 when the fragrance card element 12 is in the retracted position. The fragrance sampler may contain a single cover element 20, for example as illustrated in FIG. 1, from behind which the fragrance card is extended, or, may contain multiple cover elements 20 for example as illustrated in FIG. 2, with multiple openings through which multiple fragrance card elements 12 are extended during use.

The frame 26 may be may be made of paper, paperboard, polymers, or preferable cardstock, and may attach or contain one or more cover elements 20. The frame 26 together with the cover element 20 provides a space 28 to contain the fragrance card 10. The space 28 between the inner surfaces of the frame and the cover element second side 24 provide containment for the fragrance card 10 and also guide the fragrance card 10 during movement from the retracted to the extended position. The frame 26 retains the relative positions the fragrance card 10, fragrance card element 12, and the cover element 20 such that the microencapsulated fragrance 16 is in contact with the second side 24 of the cover element 20 in the retracted position, and ensures that friction is generated between the microencapsulated fragrance 16 and the cover element 20 when the fragrance card 10 is moved from the retracted to the extended position. The frame 26 may enclose any or all surfaces of the fragrance card elements 12 that are not covered by the cover elements 20. The frame 26 may overlap or be integrated with the cover element 20, and/or contain additional structures, for example a window 30 through which the fragrance card elements 12 are exposed when in the extended position, as illustrated in FIG. 2. The frame 26 may also contain ornamental designs or means for attaching the Fragrance Slide Sampler to the direct mailer or magazine used to deliver the device. The frame and cover element may be made or assembled from one or more parts or subassemblies using adhesive materials 32 known in the art, for example glues or fasteners. The frame 26 may employ any means of attachment to the cover element 20 including adhesive materials 32, and/or may be integrated in a continues design, for example, where the frame 26 and cover element 20 are fabricated out of a continuous piece of cardstock and folded at predetermined positions 34 to create the combined structure as illustrated in FIGS. 1 and 2. The frame 26 and cover element 20 may be of a continuous material whereby the terms cover element and frame defines functional regions of the same material. The frame may also contain a means of stopping the movement of the fragrance card to prevent the fragrance card from separating from the Samper.

From the aforementioned description, a Fragrance Slide Sampler device has been described. The Fragrance Slide Sampler device is uniquely capable of retaining a fragrance during transport to a consumer and easily disperses the fragrance upon use. Alternatively embodiments of the Fragrance Slide Sampler are envisioned for example whereby the microencapsulated fragrance may be applied to the other surfaces of the device providing that movement and friction expose and abrade the microencapsulated fragrance and release the fragrant oils. It is also envisioned that a means for moving may be placed on the frame or cover elements instead of the fragrance card provided that the user is provided with a means for exposing the fragrance card element and microencapsulated fragrance.

These together with other objects of the invention, along with the various features of novelty that characterize the invention, are pointed out with particularity in the claims annexed to and forming a part of this disclosure. For a better understanding of the invention, its operating advantages, and the specific objects attained by its uses, reference should be had to the accompanying drawings and descriptive matter in which there is illustrated a preferred embodiment of the invention.

We claim:

1. A fragrance sampler comprising:
a fragrance card comprising one or more fragrance card elements with at least one surface comprising a microencapsulated fragrance;
one or more cover elements comprising a first side and a second side, said first side being visible to a user and exposed to the atmosphere, said second side being in contact with the microencapsulate surface, said cover element substantially concealing the fragrance card from the atmosphere and from view;
a frame attached to said cover element providing a space between said second side and said frame, for containment of said fragrance card, whereby said microencapsulate surface is in contact with said second side; and
a means for moving the fragrance card relative to the one or more cover elements whereby moving the fragrance card exposing the fragrance card to the atmosphere and the user, whereby fragrance oils are released.

2. The fragrance sampler of claim 1, wherein the fragrance card, cover element or and frame are comprised of cardstock.

3. The fragrance sampler of claim 1, wherein the fragrance card comprises a plurality of fragrance card elements and a corresponding number of cover elements.

5. The fragrance sampler of claim 1, wherein release of the fragrant oils is caused by friction between the second side of said cover element and the microencapsulated fragrance.

6. The fragrance sampler of claim 1, wherein one or more fragrance card elements contain printed words or deigns that may be seen by the user when one or more fragrance card elements are in the extended position.

7. The fragrance sampler of claim 1, further comprising a means for moving the fragrance card wherein said means for moving the fragrance card is operatively attached to the fragrance card and operatively attached to the frame.

8. The fragrance sampler of claim 1, wherein said frame and the one or more cover elements direct the movement of the fragrance card against the second side of the cover element when the means for moving the fragrance card relative to the one or more cover elements moves the fragrance card exposing the fragrance card to the atmosphere and the user.

9. The fragrance sampler of claim 1, wherein said second side comprises an abrasive element.

10. The fragrance sampler of claim 1, wherein the microencapsulated fragrance form an adhesion between the fragrance card and the second side of the cover element.

11. A fragrance sampler comprising:
a fragrance card with at least one surface comprising a contained fragrance;
a cover element substantially concealing the fragrance card from the atmosphere and from view;
a frame attached to said cover element providing a space between said cover element and said frame for containment of said fragrance card; and
a means for moving the fragrance card relative to the cover element, operatively attached to the fragrance card and operatively attached to the frame where by movement of said means for moving the fragrance card results exposing the fragrance card to the atmosphere and the user.

12. The fragrance sampler of claim 11, wherein the fragrance card, cover element or and frame are comprised of cardstock.

13. The fragrance card sampler according to claim 11, wherein the contained fragrance is attached to the fragrance card using a non-permanent adhesive.

14. The fragrance card sampler according to claim 11, wherein the contained fragrance consists of a fragrance treated material.

15. The fragrance card sampler according to claim 11, wherein the contained fragrance consist of a fragrance pouch.

16. The fragrance card sampler according to claim 11, wherein the fragrance pouch comprises a first pouch wall and a second pouch wall attached near their periphery by a non-permanent adhesive, said first pouch wall, said second pouch and said non-permanent adhesive forming an impervious barrier and an internal chamber containing a fragrance.

17. A method of manufacturing the fragrance card sampler comprising:
applying the microencapsulated fragrance to one or more of the fragrance card, fragrance card element, or the second side of the cover element;
assembling the fragrance card sampler before the microencapsulated fragrance is becomes affixed to the surface; and
whereby microencapsulated fragrance forms an adhesion between the fragrance card and the second side of the cover element.
